(19) United States
(12) Patent Application Publication
(10) Pub. No.: US 2003/0198686 A1
(43) Pub. Date: Oct. 23, 2003

(54) Chinese medicinal composition

(76) Inventor: Yongzhong Li, Si Chuan Province (CN)

Correspondence Address: BAKER & BOTTS, 30 ROCKEFELLER PLAZA, NEW YORK, NY, 10112

(21) Appl. No.: 10/197,017

(22) Filed: Jul. 16, 2002

(30) Foreign Application Priority Data
Apr. 17, 2002 (CN) 02113619.X

(51) Int. Cl. A61K 35/12; A61K 35/78

(52) U.S. Cl. 424/520; 424/725

(57) ABSTRACT

A Chinese medicinal composition useful in preventing and treating cancer comprises a composition of either Group A or Group B. Group A comprises: 5-90.5% powder of head of soft-shelled turtle; 0.5-5% powder of musk; 2-20% powder of bear gall; 5-60% powder of ageratum; and 2-10% powder of rhubarb. Group B comprises: 5-90.5% of tail of soft-shelled turtle; 5-60% of powder of Sichuan fritillary bulb; 2-20% of powder of bear gall; 2-10% powder of rhubarb; and 0.5-5% of powder of musk. The Group A and Group B compositions are taken alternatively by the patient each time. The ingredients used in this invention may be easily obtained, have a low cost, and a notable effect.

### CROSS-REFERENCE TO RELATED APPLICATIONS

This application claims priority from Chinese Patent Application No. 02113619.X, filed on Apr. 17, 2002 in the Peoples' Republic of China, which is incorporated herein by reference in its entirety.

### BACKGROUND OF THE INVENTION

1. Field of the Invention

This invention relates to a Chinese medicinal composition, in particular a composition for use in the prevention and treatment of cancer.

2. Background Information

Currently, the treatment of cancer mainly adopts Western medicinal methods, including surgery and radiotherapy. These methods bring great harm to the body but with little effect; in addition, they are simply remedial measures after diagnosis, and cannot prevent oncongenesis of cancer in advance.

The purpose of this invention is to offer a type of Chinese medicine that can prevent and treat cancer, the ingredients of which can be obtained easily and cheaply, which is safe to the body and which has a notable effect in the prevention and treatment of cancer.

### SUMMARY OF THE INVENTION

The Chinese medicine composition of this invention comprises a composition selected from either Group A or Group B. Group A is as follows:

(a) 5-90.5 wt. % of a powder comprising the head of a soft-shelled turtle;

(b) 0.5-5 wt. % of a powder comprising musk;

(c) 2-20 wt. % of a powder comprising bear gall;

(d) 5-60 wt. % of a powder comprising ageratum; and

(e) 2-10 wt. % of a powder comprising rhubarb.

Group B is as follows:

(a) 5-90.5 wt. % of a powder comprising the tail of a soft-shelled turtle;

(b) 5-60 wt. % of a powder comprising Sichuan fritillary bulb;

(c) 2-20 wt. % of a powder comprising bear gall;

(d) 2-10 wt. % of a powder comprising rhubarb; and

(e) 0.5-5 wt. % of a powder comprising musk.

### DETAILED DESCRIPTION OF THE INVENTION

The main ingredients of Group A and their weight proportion in the composition of this invention are as follows:

| | |
|---|---|
| Powder of head of soft-shelled turtle | 5-90.5% |
| Powder of musk | 0.5-5% (used at normal ambient |
| | temperature) |
| Powder of bear gall | 2-20% |
| Powder of ageratum | 5-60% |
| Powder of rhubarb | 2-10% |

The main ingredients of Group B and their weight proportion in the composition of this invention are as follows:

| | |
|---|---|
| Powder of tail of soft-shelled turtle | 5-90.5% |
| Powder of Sichuan fritillary bulb | 5-60% |
| Powder of bear gall | 2-20% |
| Powder of rhubarb | 2-10% |
| Powder of musk | 0.5-5% (used at normal ambient |
| | temperature) |

In the invention, musk has the efficacy to ventilate and smooth the aperture of the heart blocked by pathogenic factors, and can smooth the channels and collaterals, open all holes, transgress skin and bones, warm the water viscera, dispel noxious factors, remove toxic agents, kill parasites, and also has a function in abortion.

Bear gall has the function of purging heat. It can also dispel pathogenic heat from heart, subdue hyper-function of the liver, improve acuity of vision, kill parasites, cure the epilepsy due to fright and uncontrolled overflow of the five emotions.

Head of soft-shelled turtle can ventilate and smooth the channels and collaterals, and disperse accumulation of pathogen. It can also help other medicines reach the illness at the affected site, eliminate ulcers and abscesses, relieve pain and exclude purulence.

Rhubarb can clear away heat and toxic material, promote peristalsis, prevent astriction, and permit free movement of the bowels.

Ageratum can promote functional activities, normalize the function of the middle-abdomen (i.e. stomach and spleen), dispel noxious factors and eliminate dampness.

Sichuan fritillary bulb can provide moisture to the lungs, disperse accumulation of pathogen, relieve cough and reduce sputum.

Turtle tail can relieve the cold and eliminate dampness.

This invention can be made into a powder, bolus, tablet, aqueous solution, absorbent or the like. This invention is preferably taken orally.

Patients using this invention should take only one of Group A or Group B each time, in a concentration within the range of about 2.5% (2.5 g) to about 85% (85 g), based upon the total weight of the dosage taken.

Fully mixed, each group can be in the form of a powder, bolus, tablet, or after boiling and filtration, in the form of an aqueous solution. The user will take Group A or Group B alternately, as required.

The ingredients used in this invention may easily be obtained, have low cost and notable effect.

The invention claimed is:

1. A Chinese patent medicine for cancer prevention and treatment, wherein bodies and tails of soft-shelled turtles are removed and heads of said turtles are kept as the ingredient of Chinese traditional medicine group A.

2. The ingredients and their weight proportion of group A are: (unit is gram)
(a) 5-90.5% powder of head of soft-shelled turtle
(b) 5-60% powder of ageratum
(c) 2-20% powder of bear gall
(d) 2-10% powder of rhubarb
(e) 0.5-5% powder of musk (in normal temperature)

3. The Chinese patent medicine for cancer prevention and treatment as defined in claim 1, wherein heads and bodies of soft-shelled turtles are removed and tails of said turtles are kept as the ingredient of Chinese traditional medicine group B.

4. The ingredients and their weight proportion of group B are: (unit is gram)
(a) 5-90.5% powder of tail of soft-shelled turtle
(b) 5-60% powder of Sichuan fritillary bulb
(c) 2-20% powder of bear gall
(d) 2-10% powder of rhubarb
(e) 0.5-5% powder of musk (in normal temperature)

5. The medicine as defined in claim 2 or group A and the medicine as defined in claim 4 or group B should be taken alternatively every time, while another is taken next time.

6. Group A and B of the Chinese patent medicine for cancer prevention and treatment as defined in claim 2 and 4, wherein all ingredients are mixed together in proportion, and then made to powder, bolus, tablet, or after adding an amount of water, boiling by slow fire and filtration, to aqua.
